(19) United States
(12) Patent Application Publication
(10) Pub. No.: US 2018/0051249 A1
(43) Pub. Date: Feb. 22, 2018

(54) METHODS FOR MAKING NEURAL STEM CELLS AND USES THEREOF

(71) Applicant: The University of North Carolina at Chapel Hill, Chapel Hill, NC (US)

(72) Inventors: Shawn D. HINGTGEN, Chapel Hill, NC (US); Raluca DUMITRU, Chapel Hill, NC (US); Julio RODRIGUEZ BAGO, Chapel Hill, NC (US)

(21) Appl. No.: 15/554,785

(22) PCT Filed: Mar. 3, 2016
(86) PCT No.: PCT/US2016/020649

§ 371 Date: Aug. 31, 2017

Related U.S. Application Data
(60) Provisional application No. 62/128,247, filed on Mar. 4, 2015.

(51) Int. Cl. C12N 5/0797; C12N 15/86; A61K 35/30

(52) U.S. Cl. CPC C12N 5/0623; C12N 15/86; A61K 35/30; C12N 2510/00; C12N 2506/09; C12N 2501/602; C12N 2501/727; C12N 2740/15043

(57) ABSTRACT

Provided herein according to some embodiments is a method for producing an induced neural stem cell (iNSC), which method may include one or more of the steps of: providing a somatic cell; introducing into (e.g., by transfecting or transducing) said somatic cell with a nucleic acid encoding Sox2, whereby said cell expresses Sox2; and then transdifferentiating said somatic cell (e.g., by growing the cell in a neural progenitor medium), to thereby make said induced neural stem cell. In some embodiments, the transdifferentiating is carried out for a time of from 1 to 10 days, from 1 to 5 days, from 1 to 3 days, or from 12 to 24 or 48 hours.

### RELATED APPLICATIONS

This application claims the benefit of U.S. Provisional Patent Application Ser. No. 62/128,247, filed Mar. 4, 2015, the disclosure of which is incorporated by reference herein in its entirety.

### BACKGROUND

Cancers of the brain remain among the most challenging tumors to treat. Over 10,000 patients are diagnosed each year with glioblastoma (GBM), the most common primary brain tumor. GBM is typically treated with surgery and chemo-radiation therapy, but unfortunately under current treatment options the disease is usually fatal. The average time to recurrence is only 6 months, and GBM patients only survive an average of 12-15 months.

Neural stem cells (NSCs) have a unique inherent capacity to home to solid and diffuse GBM deposits, and NSCs engineered with various cytotoxic agents have been shown to reduce GBM xenografts by 70-90% while significantly extending the survival of tumor-bearing mice. However, there are minimal numbers of NSCs naturally present in the brain, and they reside deep within the cortex.

The emergence of cellular reprogramming has opened new avenues in cell therapies, but suffers from limitations. For example, the de-differentiation of a fibroblast into an induced pluripotent stem cell (iPSC) and then re-differentiation to the desired therapeutic cell type is a time-consuming process, the efficiency of iPSC generation is low, and significant safety concerns remain regarding the formation of cancerous teratomas by transplanted iPSCs or derivatives.

Transdifferentiation (TD) is a method in which cells are directly converted to differentiated somatic cells of a different lineage without passing through an intermediate iPSC stage. This direct conversion by TD obviates the safety concerns associated with the iPSC state and allows faster generation of the desired therapeutic cell type.

Neural stem cells have been created by TD, termed induced neural stem cells (iNSCs). In 2012, Matsui et. al. demonstrated h-iNSCs generation could be accomplished in 20 days by partially reprogramming human fibroblasts towards iPSCs using the four Yamanaka factors. h-iNSC generation was also achieved by expressing Sox2 in fibroblasts, but this strategy required culturing on specific feeder cells for 40 days to obtain h-iNSCs for expansion and passaging.

There remains a need for alternative methods that can rapidly provide engineered NSCs for use in cell-based therapies.

### SUMMARY

Provided herein, according to some embodiments, is a method for producing an induced neural stem cell (iNSC), which method may include one or more of the steps of: providing a somatic cell; introducing into (e.g., by transfecting or transducing) said somatic cell with a nucleic acid encoding Sox2, whereby said cell expresses Sox2; and then transdifferentiating said somatic cell (e.g., by growing the cell in a neural progenitor medium), to thereby make said induced neural stem cell. In some embodiments, the transdifferentiating is carried out for a time of from 1 to 10 days, from 1 to 5 days, from 1 to 3 days, or from 12 to 24 or 48 hours.

In some embodiments, the cell is not transfected or transduced with another transdifferentiation factor.

In some embodiments, the somatic cell is a fibroblast cell (e.g., a skin fibroblast cell).

In some embodiments, the method comprises transducing said somatic cell with a lentiviral vector comprising said nucleic acid encoding Sox2.

In some embodiments, the method further includes loading the induced neural stem cell with a therapeutic agent or a reporter molecule.

In some embodiments, the method further includes encapsulating the induced neural stem cell in a hydrogel or biodegradable scaffold matrix, or seeding onto a scaffold.

In some embodiments, the method further includes administering said induced neural stem cell to a subject in need thereof (e.g., a human subject). In some embodiments, the induced neural stem cell is allogeneic with respect to said subject. In some embodiments, the induced neural stem cell is syngeneic with respect to said subject. In some embodiments, the induced neural stem cell is autologous with respect to said subject. In some embodiments, the subject is in need of treatment for a brain cancer.

In some embodiments, the induced neural stem cell is autologous with respect to said subject and wherein said administering is carried out 1, 2, 3 or 4, to 7, 10, 14 or 21 days, after said providing the somatic cell.

Further provided is the use of an induced neural stem cell as taught herein for treating a brain cancer. Also provided is the use of an induced neural stem cell as taught herein in the preparation of a medicament for the treatment of a brain cancer.

### BRIEF DESCRIPTION OF THE DRAWINGS

FIG. 1. Generation and characterization of diagnostic and therapeutic iNSCs. (A) Schematic depiction of the strategy used to create therapeutic and diagnostic variants of h-iNSCs. Human fibroblasts were transduced with Sox2 and placed in NSC-inducing neural progenitor media. After 4 days, the h-iNSCs were expanded and transduced with optical reporters or tumoricidal transgenes. (B) White light and fluorescent photomicrographs of human fibroblasts and h-iNSCs grown as monolayers, neurospheres, or stained with antibodies against nestin. (C) Summary graph showing the expression of nestin at different days after induction of h-iNSC generation. (D) Immunofluorescent staining that reveals h-iNSC-GFP expression of the NSC marker nestin. GFAP+ astrocytes and Tuj1+ neurons were differentiated from h-iNSC-GFP by mitogen removal. In contrast, no staining was observed for the pluripotency markers TRA-160 or OCT4. Fluorescent images showing only the secondary antibody channel are shown in the bottom row. (E) RT-PCR analysis of Nestin, Sox2, Nanog, and OCT3/4 expression in normal human fibroblasts, h-iNSCs, and h-iPSCs.

FIG. 2. Engineered h-iNSCs home to GBM. (A) h-iNSC-GFPFL were seeded 500 μm apart from mCherry-expressing human GBM cells and placed in a fluorescence incubator microscope. Time-lapse fluorescent images were captured every 10 minutes for 24 hours and used to construct movies that revealed the migration of iNSC in real-time. (B) Summary images showing migration of h-iNSC-GFPFL or parental human fibroblasts towards U87-mCFL at 0 hrs and 24 hrs after plating. (C) Single cell tracings depicting the path of h-iNSC-GFPFL directed migration towards GBM over 24 hrs. Additional images show the limited migration of parental human fibroblasts. Dotted line indicates the site of GBM seeding. (D-F) Summary graph showing the directionality (D), distance (E), and velocity (F) of h-iNSCs or fibroblast migration towards GBM cells determined from the real-time motion analysis. (G-H) To assess h-iNSC migration to solid GBMs, U87 GBM spheroids were co-cultured with h-iNSCs in a 3D leviation system (G) Fluorescent imaging showed the migration of h-iNSC-GFPFL into U87 spheroids and their penetration towards the core of the tumor spheroid over time (H).

FIG. 3. In vivo characterization of iNSCs transplanted in the mouse brain. (A) summary graph demonstrating the proliferation of unmodified h-iNSCs and h-iNSCs engineered to express mCherry-FLuc. (B-C) h-iNSC were implanted into the frontal lobe of mice and serial bioluminescence imaging was used to monitor their persistence over 3 weeks. Summary graphs demonstrated the h-iNSCs persisted in the brain from 25 days, although they were gradually cleared (B) Immunofluorescence analysis of h-iNSCs 14 days post-implantation into the brain showed Nestin+ and Tuj+ cells, however no co-localization between h-iNSCs and the pluripotency markers Oct-4 and TRA-160 was observed (C).

FIG. 4. h-iNSC-mediated TRAIL therapy for solid GBM. (A-B) Representative fluorescent photomicrographs depicting the growth of h-iNSCs engineered to secrete the pro-apoptotic agent TRAIL and grown in a monolayer (A) or as floating neurospheres (B). (C) Images and summary data of 3D suspension cultures showing the viability of mCherry+ human U87 GBM spheroids mixed with therapeutic h-iNSC-sTR or control cells at ratio of 1:2 or 1:2. GBM spheroid viability was determined by luciferase imaging 48 hrs post-treatment. (D) h-iNSC-sTR therapy for solid GBM was performed by xenografting a mixture of h-iNSC-sTR and U87 GBM cells into the parenchyma of SCID mice. (E-F) Representative BLI images (E) and summary data (F) demonstrating the inhibition of sold U87 GBM progression by h-iNSC-sTR therapy compared to control-treated mice. (G) Kaplan-Meier curved demonstrating the extension in survival in h-iNSC-sTR-treated animal compared to h-iNSC-control. (H) Representative images demonstrating the expression of cytotoxic, differentiation, and pluripotency markers in h-iNSC-sTR following therapy. A subset of animals were sacrificed 14 days after therapy, and brain sections were stained with antibodies against nestin, TRAIL, GFAP, Tuj-1, Oct-4, or TRA-160 and the co-localization between staining and GFP+h-iNSC-sTR was visualized.

FIG. 5. h-iNSC prodrug/enzyme therapy for human patient-derived GBMs. (A-D) The anti-tumor effects of h-iNSC-TK therapy were determined in two different 3D culture models. h-iNSC-TK were either mixed with GFP+ GBM4 patient-derived GBM cells (A, B) or seeded adjacent to established GBM4 spheroids (C, D) and GCV was added to initiate tumor killing. Serial fluorescent images showed the time-dependent decrease in GBM4 spheroid volume by h-iNSC-TK/GCV therapy. (E) Summary graph demonstrating the reduction in GBM4 spheroid volume over 7 days by h-iNSC-TK/GCV therapy. (F-J) h-iNSC-TK therapy was assessed in vivo by injecting h-iNSC-TK cells into GBM4 tumors established 10 days earlier in the brain of mice (F). Serial BLI showed the progression of GBM4 tumors was significantly inhibited by h-iNSC-TK/GCV therapy (G). Kaplan-Meier survival curves demonstrating the survival of mice bearing GBM4 tumors treated with h-iNSC-TK/GCV therapy or control h-iNSCs (H). (I-J) Representative whole-brain and high-magnification images showing GBM4 volumes and h-iNSC-TK distribution 21 days after delivering h-iNSC-control (I) or h-iNSC-TK (J) into established GBM4 tumors. A large GBM4 tumor was present in the control-treated animals and only a small GBM4 foci was detected in the h-iNSC-TK-treated brain.

FIG. 6. Intracavity h-iNSC-TK therapy for surgically resected diffuse GBMs. (A-C) 3D suspension cultures were used to determine the migration and anti-tumor efficacy of synthetic extracellular matrix (sECM)-encapsulated h-iNSC-TK against patient-derived GBM8 spheroids (A). h-iNSC-TK encapsulated in sECM were found to migrate from the matrix and populate GBM8 spheroids 3 days after seeding (B). Representative images and summary data demonstrated that h-iNSC-TK encapsulated in sECM significantly reduce the volume of GBM8 spheroids compared to control-treated spheroids (C). (D) To mimic clinical h-iNSC therapy for surgically resected GBM, h-iNSC-TK were encapsulated in sECM and transplanted into the surgical cavity following resection of diffuse patient-derived GBM8 tumors expressing mCherry-FLuc. (E) Representative images and summary data of serial imaging demonstrating the significant inhibition in tumor recurrence following intra-cavity h-iNSC-TK therapy for post-operative minimal GBM8 tumors. (F) Kaplan-Meier survival curves of mice that underwent surgical resection of diffuse GBM8 patient-derived tumor cells treated with control h-iNSC or h-iNSC-TK encapsulated in sECM and transplanted into surgical cavity.

### DETAILED DESCRIPTION

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

A “neural stem cell” as used herein refers to a multipotent cell capable of differentiating into central nervous system cells such as neurons, astrocytes and/or oligodendrocytes.

“Transdifferentiation” or “transdifferentiating” is a method in which differentiated somatic cells are directly converted to differentiated or multipotent somatic cells of a different lineage without passing through an intermediate pluripotent stem cell (iPSC) stage. Transdifferentiation may be carried out by exposing the cells to one or more transdifferentation factors and/or growing the cells in a medium that promotes transdifferentiation into the desired cell type. Monitoring the transdifferentiation may be performed using methods known in the art, such as monitoring marker expression indicative of differentiated somatic cells and/or stem cells.

Differentiated somatic cells may be collected from any accessible source, such as tissue, bodily fluids (e.g., blood, urine), etc. In some embodiments, the somatic cell is a fibroblast cell such as a skin fibroblast cell. For example, skin cells may be collected from the border of a surgical incision, e.g., during an accompanying surgical procedure, or using a traditional skin punch as a stand-alone procedure. Skin could be collected from any area, including, but not limited to, collection from the scalp or forearm.

In some embodiments as taught herein, the transdifferentiating is carried out for a time of from 1, 2, or 3 to 8, 9 or 10 days, from 1, 2 or 4 to 5, 6 or 7 days, from 1 or 2 to 3 days, or from 12 to 24, 48 or 72 hours.

“Transdifferentiation factor” as used herein is a protein such as a transcription factor that promotes the direct conversion of one somatic cell type to another. Examples include, but are not limited to, Oct4, Sox2, Klf4, Myc, Asc11, Brn2, Myt11, Olig2, Zic1, etc. In some embodiments, the method of transdifferentiation is a single-factor transdifferentation in that only one transdifferentiation factor is used.

“Sox2” is a member of the Sox family of transcription factors and is expressed in developing cells in the neural tube as well as in proliferating progenitor cells of the central nervous system. In some embodiments, Sox2 is used as the transdifferentiation factor in the methods taught. In some embodiments, Sox2 is used to carry out a single-factor transdifferentiation.

“Nestin” is expressed predominantly in stem cells of the central nervous system, and its expression is typically absent from differentiated central nervous cells. “GFAP” or “glial fibrillary acidic protein,” is an intermediate filament protein expressed by central nervous system cells, including astrocytes. “Tuj-1” or “βIII tubulin” is a neural marker.

“Nanog” and “OCT3/4” are known stem cell markers.

In some embodiments as taught herein, the transdifferentiating is carried out without the use of feeder cells, e.g., in a neural progenitor medium. Feeder cells, as known in the art, are additional cells grown in the same culture dish or container, often as a layer (e.g., a mouse fibroblast layer on the culture dish) to support cell growth.

“Neural progenitor medium” as used herein is a medium or media that promotes the transdifferentiation (TD) of somatic cells into neural stem cells (“induced” neural stem cells). In some embodiments, the neural progenitor medium includes one or more ingredients selected from: a cell culture medium containing growth-promoting factors and/or a nutrient mixture (e.g., DMEM/F12, MEM/D-valine, neurobasal medium etc., including mixtures thereof); media supplements containing hormones, proteins, vitamins and/or amino acids (e.g., N2 supplement, B27 supplement, non-essential amino acids (NEAA), L-glutamine, Glutamax, BSA, insulin, all trans retinoic acid, etc. including mixtures thereof); and optionally small molecule inhibitors (e.g., SB431542 (BMP inhibitor), LDN193189 (TGF-β1 inhibitor), CHIR99021 (GSK3β inhibitor), etc., including mixtures thereof). Ingredients may also include one or more of beta-mercaptoethanol, transferrin; sodium selenite; and cAMP. Suitable concentrations of each of these ingredients are known to those of skill in the art and/or may be empirically determined. Example concentrations of ingredients is also provided in Example 2 below. In some embodiments, the neural progenitor medium is a premade medium, such as STEMdiff™ Neural Induction Medium (STEMCELL™ Techologies, Vancouver, British Columbia, Canada).

In some embodiments, neural stem cells are loaded with TERT (telomerase reverse transcriptase) to promote their lifespan and/or enhance their ability to be expanded by cell culture. In some embodiments, the TERT is human telomerase reverse transcriptase (“hTERT”).

“Treat” or “treatment” as used herein refers to any type of treatment that imparts a benefit to a patient afflicted with a disease or disorder such as a cancer, neurodegenerative disorder or neural trauma, including improvement in the condition of the patient (e.g., in one or more symptoms), delay in the progression of the disease, delay in onset or recurrence of the disease, etc.

The present invention is primarily concerned with the treatment of human subjects, but the invention may also be carried out on animal subjects, particularly mammalian subjects such as mice, rats, dogs, cats, livestock and horses for veterinary purposes, and for drug screening and/or drug development purposes. Subjects may be of any age, including infant, juvenile, adolescent, adult, and geriatric subjects. In some embodiments, induced neural stem cells are allogeneic or autologous with respect to the subject.

Cancers to be treated include brain cancers, which may be primary or secondary brain cancer. A “primary brain cancer” is an intracranial cancer of central nervous system cells. Types of brain cancer include, but are not limited to, gliomas (e.g., glioblastoma or glioblastoma multiforme (GBM)), meningiomas, medulloblastomas, pituitary adenomas and nerve sheath tumors. A “secondary brain cancer” is a cancer located in the central nervous system that includes cells metastasized from other areas of the body, e.g., breast cancer, melanoma, lung cancer, prostate cancer, etc.

In some embodiments, neural stem cells as taught herein are loaded with (i.e., contain) a therapeutic agent, a reporter molecule and/or a nucleic acid capable of expressing the same. In some embodiments, the therapeutic agent is a protein toxin (e.g., a bacterial endotoxin or exotoxin), an oncolytic virus (e.g., a conditionally replicative oncolytic adenovirus, reovirus, measles virus, herpes simplex virus (e.g., HSV1716), Newcastle disease virus, vaccinia virus, etc.), or a pro-apoptotic agent (e.g., secretable tumor necrosis factor (TNF)-related apoptosis-inducing ligand (S-TRAIL)). See, e.g., WO 19940/16718 to Weiss et al; WO 2014/018113 to Shah et al.; WO 2009/148488 to Martuza et al.; US 2009/0175826 to Subbiah et al.

In some embodiments, the therapeutic agent is a pro-inflammatory protein such as an interleukin, cytokine, or antibody.

In some embodiments, the therapeutic agent is an enzyme useful for enzyme/prodrug therapies (e.g., thymidine kinase (e.g., with gancyclovir prodrug), carboxylesterase (e.g., with CTP-11), cytosine deaminase, etc.).

In some embodiments, the therapeutic agent is an RNAi molecule such as miRNA or siRNA.

In some embodiments, the neural stem cells are loaded with nanoparticle/drug conjugates.

Reporter molecules are known in the art and include, but are not limited to, Green Fluorescent Protein, β-galactosidase, alkaline phosphatase, luciferase, and chloramphenicol acetyltransferase gene. See, e.g., US 2013/0263296 to Pomper et al.

Loading of the neural stem cells may be accomplished using art-known methods, such as transfecting the cells with a nucleic acid capable of producing a therapeutic or reporter protein, transducing the cells with a viral vector, lipid-based or polymeric loading of the cells with a therapeutic agent and/or reporter molecule, etc.

“Transfecting” is the transfer of heterologous genetic material into a cell, often through the use of a vector (i.e., molecule used as a vehicle to carry foreign genetic material into another cell). Methods of transfecting eukaryotic cells are known, and may include, but are not limited to, electroporation, use of cationic liposome based reagents, nanoparticle polymer liposomes, etc.

“Transducing” is the transfer of heterologous genetic material into a cell by means of a virus. Such viral vectors are known and may include, but are not limited to, lentiviral vectors, adenoviral vectors, etc.

Administration of the neural stem cells may be performed using methods known in the art. For example, intracranial administration of the cells may be performed for the treatment of a brain cancer, preferably intratumoral administration or intracavity administration performed after surgical removal of at least a part of a brain tumor.

In some embodiments, the cells are encapsulated by a matrix such as a hydrogel matrix (e.g., a synthetic extracellular matrix) and/or seeded onto a scaffold, which may then be administered or implanted, e.g., intracranially. See, e.g., PCT patent application publication WO 2014/035474 to Shah; US 2014/0086907 to Shah, which are each incorporated by reference herein.

The present invention is explained in greater detail in the following non-limiting examples.

### EXAMPLES

### Example 1: Rapid Transdifferentiation of Human Skin Cells

The ability to rapidly generate h-iNSCs from human skin may enable patient-specific therapies to treat cancer. The efficiency of iNSC generation is significantly higher than other cellular reprogramming strategies, suggesting large numbers of h-iNSCs could be generated from small amounts of skin. Patient-specific derivation could avoid immune rejection to maximize tumor killing and for treatment durability.

Cell-based drug carriers must be generated quickly in order to treat patients with rapidly progressing cancers, and h-iNSCs can be created in weeks. Also, unlike iPSCs, h-iNSCs do not form teratomas after transplant.

In this study, the potential of TD-derived h-iNSC therapies was investigated as autologous GBM therapy for human patients. These methods are capable of converting human skin into h-iNSCs 6-fold faster than previous methods, which is significant because time is a priority for GBM patient therapy. This strategy was used to create the first h-iNSCs engineered with cytotoxic agents and optical reporters. A combination of real-time molecular imaging, 3-D cell culture, and multiple human GBM xenografts models were used to investigate the fate, tumor-specific homing, and efficacy of h-iNSC therapy against solid and surgically resected GBM.

### Materials and Methods

Cell Lines:

U87, GBM8, GBM4, 293T, and human fibroblast cells (CCD-1099Sk, others) were grown as previously described (Hingtgen et al., Stem Cells 28, 832-841, 2010; Wakimoto et al., Cancer Res 69, 3472-3481, 2009). Lentiviral vectors (LV) encoding hTERT and Sox2 were purchased from Addgene (Cambridge. Mass., USA). All cDNA were under control of the tetracycline promoter.

Human iNSCs (h-iNSC) were generated following a single-factor Sox2 and feeder-free method. Briefly, 200,000 human fibroblasts were seeded in 6-well plates and transduced with the LV cocktail containing hTERT and Sox2 in media containing protamine sulfate (5 μg/ml, Sigma). Two days after infection, the media was changed to STEMdiff™ Neural Induction Medium (STEMCELL Technologies, Vancouver, Canada) containing doxycycline (10 μg/ml, Sigma, St. Louis, Mo., USA). Media was changed every 3 days. Neurosphere formation was induced by culturing in low-adherent flasks.

Lentiviral Vectors:

In addition to the reprogramming vectors, the following lentiviral vectors were used in this study: LV-GFP-FL, LV-GFP-RLuc, LV-mC-FL, LV-sTR, LV-diTR and LV-mRFP-hRLuc-ttk. GFP-RLuc and GFP-FL were constructed by amplifying the cDNA encoding Renilla luciferase or firefly luciferase using the vectors luciferase-pcDNA3 and pAC-hRluc (Addgene), respectively. The restriction sites were incorporated in the primers, the resulting fragment was digested BglII and SalI, and ligated in frame in BglII/SalI digested pEGFP-C1 (Clontech, Mountain View, Calif., USA). The GFP-FL or GFP-RLuc fragments were digested with Agel (blunted) and SalI, and ligated into pTK402 (provided by Dr. Tal Kafri, UNC Gene Therapy Center) digested BamHI (blunted) and XhoI to create LV-GFPFL or LV-GFP-RLuc. Similarly, mCFL was created by amplifying the cDNA encoding firefly luciferase from luciferase-pcDNA3, ligating into BglII/SalI digested mCherry-C1 (Clontech), and ligating the mC-FL fragment into pTK402 LV backbone using blunt/XhoI sites. To create LV-sTR and LV-diTR, the cDNA sequence encoding sTR or diTR was PCR amplified using custom-synthesized oligonucleotide templates (Invitrogen, Carlsbad, Calif., USA). The restriction sites were incorporated into the primers, the resulting fragment was digested with BamH1 and XhoI, and ligated in-frame into BamH1/XhoI digested pLVX plasmid. Both LV-sTR and LV-diTR have IRES-GFP (internal ribosomal entry sites-green fluorescent protein) elements in the backbone as well as CMV-driven puromycin element. All LV constructs were packaged as LV vectors in 293T cells using a helper virus-free packaging system as described previously (Sena-Esteves et al., Journal of virological methods 122, 131-139, 2004). h-iNSCs and GBM cells were transduced with LVs at varying multiplicity of infection (MOI) by incubating virions in a culture medium containing 5 μg/ml protamine sulfate (Sigma) and cells were visualized for fluorescent protein expression by fluorescence microscopy.

Cell Viability and Passage Number:

To assess the proliferation and passage number of modified and unmodified h-iNSCs, h-iNSCs expressing GFP-FL, sTR or unmodified cells were seeded in 96-well plates. Cell viability was assessed 2, 3, 4, 5, 8, and 10 days after seeding using CellTiter-Glo® luminescent cell viability kit (Promega). Maximum passage number was assessed by monitoring the number of times iNSCs, iNSC-sTR, or WT-NSC were subcultured without alterations in morphology, growth rate, or transduction efficiency.

Immunohistochemistry and In Vitro Differentiation:

To determine the effects of LV modification on h-iNSC differentiation, h-iNSCs were transduced with LV-GFP-FL or LV-sTR. Engineered or unmodified cells were fixed, permeabilized, and incubated for 1 h with anti-nestin Polyclonal antibody (Millipore, MAB353, 1:500, Billerica, Mass., USA). Cells were washed and incubated with the appropriate secondary antibody (Biotium, Hayward, Calif., USA) for 1 hr. Cells were then washed, mounted, and imaged using fluorescence confocal microscopy. For differentiation, engineered or non-transduced h-iNSCs were cultured for 12 days in N3 media depleted of doxycycline, EGF, and FGF. Cells were then stained with antibodies directed against nestin, glial fibrillary acidic protein (GFAP; Millipore, MAB3405, 1:250), or Tuj-1 (Sigma, T8578, 1:1000) and detected with the appropriate secondary antibody (Biotium). Nuclei were counterstained with Hoechst 33342 and the results analyzed using a FV 1200 laser confocal microscope (Olympus, Center Valley, Pa.).

Three-Dimensional Tissue Culture.

Three-dimensional levitation cell cultures were performed using the Bio-Assembler Kit (Nano3D Biosciences, Houston, Tex.). Confluent 6 well plates with GBM or h-iNSC were treated with a magnetic nanoparticle assembly (8 μl cm⁻² of cell culture surface area or 50 μl ml⁻¹ medium, NanoShuttle (NS), Nano3D Biosciences) for overnight incubation to allow for cell binding to the nanoparticles. NS was fabricated by mixing iron oxide and gold nanoparticles cross-linked with poly-1-lysine to promote cellular uptake. (Souza, G. R., et al. Three-dimensional tissue culture based on magnetic cell levitation. Nat Nanotechnol 5, 291, 2010). Treated GBM and h-iNSC were then detached with trypsin, resuspended and mixed at different ratios (1:1 and 1:0.5) in an ultra-low attachment 6 well plate with 2 ml of medium. A magnetic driver of 6 neodymium magnets with field strength of 50 G designed for 6-well plates and a plastic lid insert were placed atop the well plate to levitate the cells to the air-liquid interface. Media containing 4 μg/ml GCV was added to the co-culture of GBM with h-iNSC expressing ttk. Fluorescence images where taken over time to track the cell viability of both populations (previously labeled with different fluorescence). For BLI of 3D cell culture, 100 μl/well of Fluc substrate stock reagent was added to the media and imaged using an IVIS Kinetic Optical System (PerkinElmer) with a 5 minute acquisition time. Images were processed and photon emission quantified using LivingImage software (PerkinElmer).

Real-Time Imaging and Motion Analysis:

Migration was assessed in novel 2-dimensional and 3-dimensional culture systems.

2-Dimensional Migration:

h-iNSCs expressing RFP were seeded in micro-culture inserts in glass bottom microwell dishes (MatTek, Ashland, Mass., USA) using 2-chamber cell culture inserts (ibidi, Verona, Wis., USA). U87 glioma cells expressing GFP were plated into the adjacent well (0.5 mm separation) or the well was left empty. 24 hrs after plating, cells were placed in a VivaView live cell imaging system (Olympus) and allowed to equilibrate. The insert was removed and cells were imaged at 10× magnification every 20 minutes for 36 hours in 6 locations per well (to monitor sufficient cell numbers) in three independent experiments. NIH Image was then used to generate movies and determine both the migrational velocity, total distance migrated, and the directionality of migration.

3-Dimensional Migration:

h-iNSC migration to GBM spheroids was assessed in 3-D culture systems by creating h-iNSC and GBM spheroids using levitation culture as described above. h-iNSC and GBM spheroids were co-cultured in levitation systems. Real-time imaging was performed to visualize the penetration of GBM spheroids by h-iNSCs in suspension.

Co-Culture Viability Assays:

mNSC expressing sTR or control GFP-RL (5×10³) were seeded in 96 well plates. 24 hrs later, U87-mC-FL, LN18-mC-FL, or GBM8-mC-FL human GBM cells (5×10³) were seeded into the wells and GBM cell viability was measured 24 hrs later by quantitative in vitro bioluminescence imaging. GBM cells were also assessed at 18 hrs for caspase-3/7 activity with a caged, caspase 3/7-activatable DEVD-aminoluciferin (Caspase-Glo 3/7, Promega, Madison, Wis., USA).

h-iNSC Survival and Fate In Vivo:

To determine the survival of h-iNSCs in vivo, h-iNSC expressing mCherry-FL (7.5×10⁶ cells/mouse) were suspended in PBS and implanted stereotaxically into the right frontal lobe of mice (n=7). h-iNSC survival was determined by serial bioluminescence imaging performed for 20 days. To determine the fate of h-iNSCs at a cellular resolution, animals were sacrificed 21 days post-implantation, brains extracted sectioned. Tissue sections were stained with antibodies against nestin, GFAP, Tuj-1, Oct-4, and TRA-160, and visualized using a secondary antibody labeled with CF™488.

Co-Culture Viability Assays:

3-D levitation culture was used in three separate in vitro cytotoxicity studies. h-iNSCs expressing 2 different cytotoxic agents were used to treat 1 established GBM cell line (U87) and 2 patient-derived GBM lines (GBM4, GBM8). 1) To determine the cytotoxicty of TRAIL therapy, h-iNSC-sTR or h-iNSC-mCherry spheroids were co-cultured in suspension with U87-GFP-FLuc spheroids at a iNSC:GBM ratio of 1:2 or 1:1. GBM spheroid viability was determined 48 hrs later by FLuc imaging. 2) To determine the cytotoxicity of pro-drug enzyme therapy for patient-derived GBMs, h-iNSC-TK spheroids were co-cultured in suspension with patient-derived GBM4-GFP-FLuc spheroids or mixed with GBM cells prior to sphere formation. Spheroids were cultured with or without gancyclovir (GCV) and GBM spheroid viability was determine 0, 2, 4, or 7 days after addition of the pro-drug by FLuc imaging. 3) To determine the cytotoxicity of sECM-encapsulated iNSC pro-drug/enzyme therapy, h-iNSC-TK were encapsulated in sECM and placed in levitation cultured with patient-derived GBM8-GFP-FLuc spheroids. Viability was determine by FLuc imaging.

Anti-GBM Efficacy of h-iNSC Therapy In Vivo:

Three different xenograft studies were performed to assess the anti-GBM effects of h-iNSC therapy. h-iNSC-sTR and h-iNSC-TK therapy was tested against solid (U87), diffused patient-derived (GBM8), and surgically resected patient-derived (GBM4) xenograft models.

1) To determine the therapeutic efficacy of h-iNSC-TRAIL against solid human U87 tumors, a combination of h-iNSC-TRAIL or iNSC-GFP-RLuc (7.5×10⁵ cells/mouse) were stereotactically implanted into the right frontal lobe of mice (n=7) together with U87-mC-FL cells (1×10⁶ cells/mouse). Therapeutic response was then determined by following tumor volumes with FL bioluminescence imaging as described previously. Briefly, mice were given an intraperitoneal injection of D-Luciferin (4.5 mg/mouse in 150 μl of saline) and photon emission was determined 5 minutes later using an IVIS Kinetic Optical System (PerkinElmer) with a 5 minute acquisition time. Images were processed and photon emission quantified using LivingImage software (PerkinElmer). Additionally, mice were followed for survival over time.

2) To investigate the efficacy of h-iNSC prodrug/enzyme therapy against invasive patient-derived GBM, mice were stereotactically implanted in the right frontal lobe with GBM8 cells expressing mC-FL (1.5×10⁵ cells/mouse). Three days later, h-iNSC-TK (n=7, 7.5×10⁵ cells/mouse) or h-iNSC-mRFP-hRLuc (n=7, 7.5×10⁵ cells/mouse) were implanted into the tumor implantation site. GCV was injected i.p. daily during two weeks at a dose of 100 mg/kg. Changes in tumor volume were assessed by FLuc imaging as described above and mice were followed for survival over time.

3) To determine the efficacy of h-iNSC therapy against post-surgical minimal GBM, image-guided GBM resection in mice was performed according to our previously reported strategy. Patient-derived GBM8-GFP-FLuc were harvested at 80% confluency and implanted stereotactically (5×10⁵ cells) in the right frontal lobe: 2 mm lateral to the bregma and 0.5 mm from the dura. Following immobilization on a stereotactic frame, mice were placed under an Olympus MVX-10 microscope. Intraoperative microscopic white light, GFP, and RFP images were captured throughout the procedure using with a Hamamatsu ORCA 03G CCD (high resolution) camera and software (Olympus). A midline incision was made in the skin above the skull exposing the cranium of the mouse. The intracranial xenograft was identified using GFP fluorescence. A small portion of the skull covering the tumor was surgically removed using a bone drill and forceps and the overlying dura was gently peeled back from the cortical surface to expose the tumor. Under GFP fluorescence, the GBM8-GFPFL tumor was surgically excised using a combination of surgical dissection and aspiration, and images of GFP were continuously captured to assess accuracy of GFP-guided surgical resection. Following tumor removal, the resulting resection cavity was copiously irrigated and the skin closed with 7-0 Vicryl suture. No procedure-related mortality was observed. All experimental protocols were approved by the Animal Care and Use Committees at The University of North Carolina at Chapel Hill and care of the mice was in accordance with the standards set forth by the National Institutes of Health Guide for the Care and Use of Laboratory Animals, USDA regulations, and the American Veterinary Medical Association. Following surgical resection, h-iNSC-TK or h-iNSC-mC-FL (5×10⁵ cells) were encapsulated in hyaluronic sECM hydrogels (Sigma) and transplanted into the post-operative GBM cavity. GBM recurrence was visualized by FLuc imaging as described above and mice were followed for survival.

Tissue Processing:

Immediately after the last imaging session, mice were sacrificed, perfused with formalin, and brains extracted. The tissue was immediately immersed in formalin. 30 μm coronal sections were generated using a vibrating microtome (Fisher Waltham, Mass., USA). For nestin, GFAP, and Tuj-1 staining, sections were incubated for 1 hr in a blocking solution (0.3% BSA, 8% goat serum, and 0.3% Triton X-100) at room temperature, followed by incubation at 4° C. overnight with the following primary antibodies diluted in blocking solution: (1) anti-human nestin (Millipore), (2) anti GFAP (Millipore), (3) anti TRAIL (ProSci, Poway, Calif.) and (4) anti-Tuj-1 (Sigma). Sections were washed three times with PBS, incubated in the appropriate secondary antibody, and visualized using a confocal microscope (Olympus).

### Results

The Rapid Transdifferentiation of Human Fibroblasts into h-iNSCs.

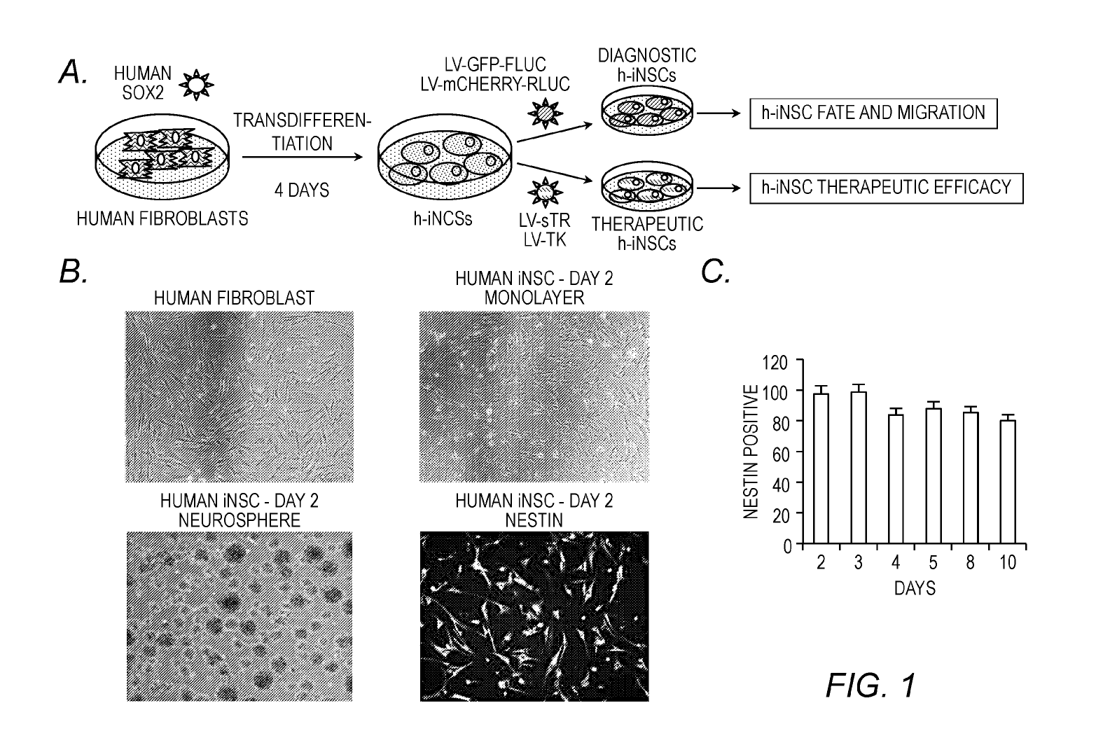

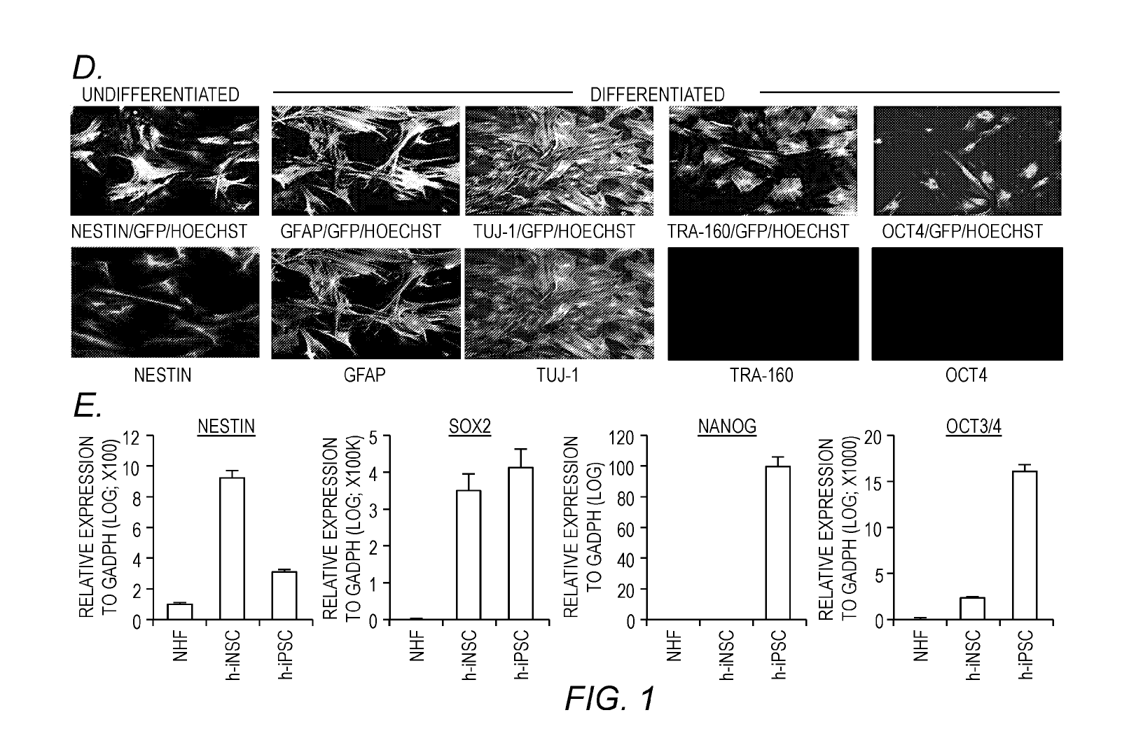

The rapid and efficient generation of h-iNSC therapies is essential for treating patients with aggressive cancer. As a new strategy, human fibroblasts were transduced with Sox2 and performed h-iNSC generation without feeder cells. Then, diagnostic h-iNSCs expressing optical reporters or therapeutic h-iNSCs expressing different cytotoxic agents were generated (FIG. 1A). First was evaluated the kinetics of generating h-iNSCs using the feeder-free/Sox2 strategy. Human fibroblasts were transduced with Sox2 and cultured in NSC-inducing media (FIG. 1B). Changes in cell morphology were observed within 48 hrs of activating Sox2 expression. Additionally, wide-spread nestin expression was detected and the h-iNSCs could form neurosphere formation. Quantification showed nestin expression in h-iNSCs remained constant from day 2 through day 10 (FIG. 1C). When induced to differentiate, the h-iNSCs expressed the astrocyte marker GFAP and the neural marker Tuj-1. Staining revealed the cells did not express the pluripotency makers TRA-160 or OCT4 (FIG. 1D). These findings were confirmed by RT-PCR analysis (FIG. 1E). The h-iNSCs showed high level of nestin expression that was absent in parental fibroblasts or human iPSC (h-iPSC). Sox2 expression was high in both h-iNSCs and h-iPSCs because Sox2 overexpression was used to generate both cell lines. Unlike h-iPSCs, h-iNSCs did not express high levels of the pluripotency markers Nanog or OCT3/4. Together, these data demonstrate the ability to create multi-potent h-iNSCs within 48 hrs using single-factor Sox2 expression.

h-iNSCs Migrate Selectively to GBM.

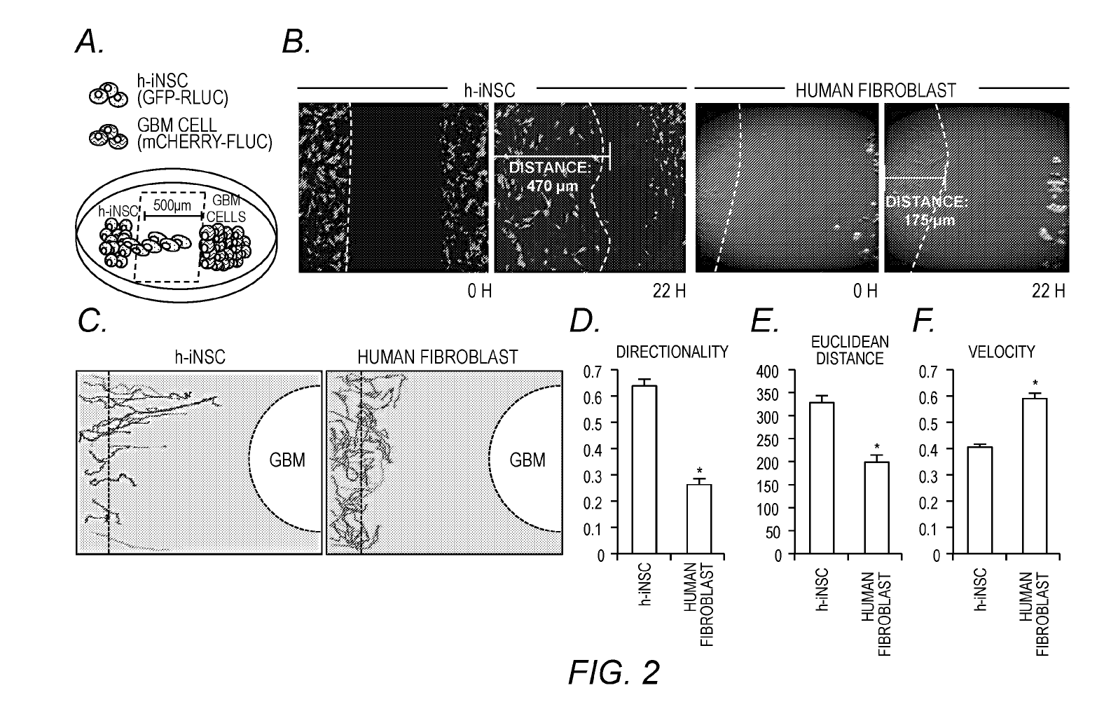

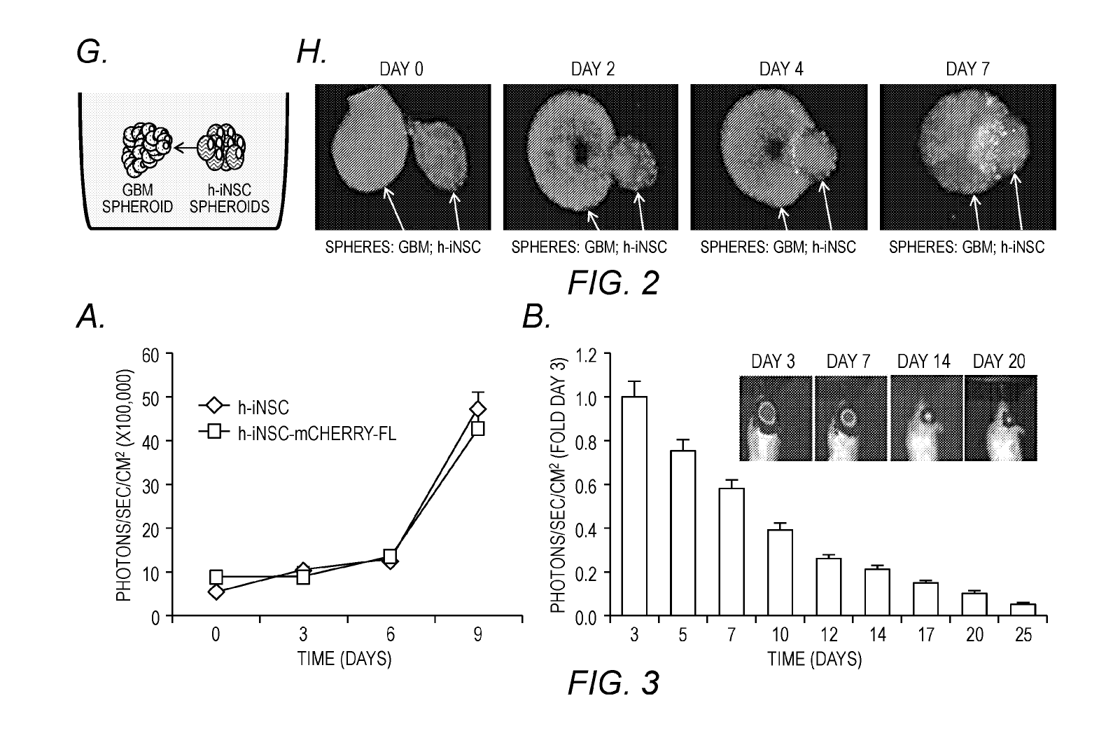

The ability to home to solid and invasive GBM deposits is one of the most beneficial characteristics of NSC-based cancer therapies. To investigate the tumor-tropic nature of h-iNSCs, we used real-time motion analysis of h-iNSCs co-cultured with human GBM cells (outlined in FIG. 2A). For reference, h-iNSC migration was compared to the parental human fibroblasts from which they were derived. It was found that h-iNSCs rapidly migrated towards the co-cultured GBM cells, covering the 500 μm gap in 22 hrs (FIG. 2B). Single cell migratory path analysis showed that the presence of GBM cells induced h-iNSC to selectively migrate towards the co-cultured GBM cells (FIG. 2C). In contrast, human fibroblasts demonstrated very little migration (FIG. 2B). Single cell migration analysis of human fibroblasts confirmed the random migratory patterns with very little displacement towards the co-cultured GBM cells (FIG. 2C). The directionality of the migration of h-iNSC was analyzed by calculating the ratio of Euclidian distance to overall accumulated distance, with perfect single direction movement yielding a ratio of 1.0 and perfectly non-directional movement yielding a ratio of 0.0. Using this analysis, we calculated an average directionality ratio that was significantly higher for h-iNSCs (0.65) than human fibroblasts (0.28) (FIG. 2D). Further analysis of single cell migration patterns demonstrated significantly increased average Euclidian distance migrated by h-iNSC (340 μm) as compared to human fibroblasts (200 μm) (FIG. 2E). The average cell velocity by h-iNSC was lower as compared to human fibroblasts (0.4 vs 0.62) (FIG. 2F). Lastly, we performed 3-D migration assays to mimic the in vivo migration of h-iNSCs into GBM foci. mCherry+h-iNSC spheroids were co-cultured with GFP+ GBM spheroids and both cell types were levitated using magnetic force (FIG. 2G). We discovered that the h-iNSCs began penetrating the GBM spheroids within hours of seeding. The h-iNSC spheroids continued to penetrate the GBM spheroids, extensively co-localizing within 8 days. Together, these observations support the conclusion that h-iNSCs possess tumoritropic properties and home to GBM cells.

h-iNSC Persistence and In Vivo Fate.

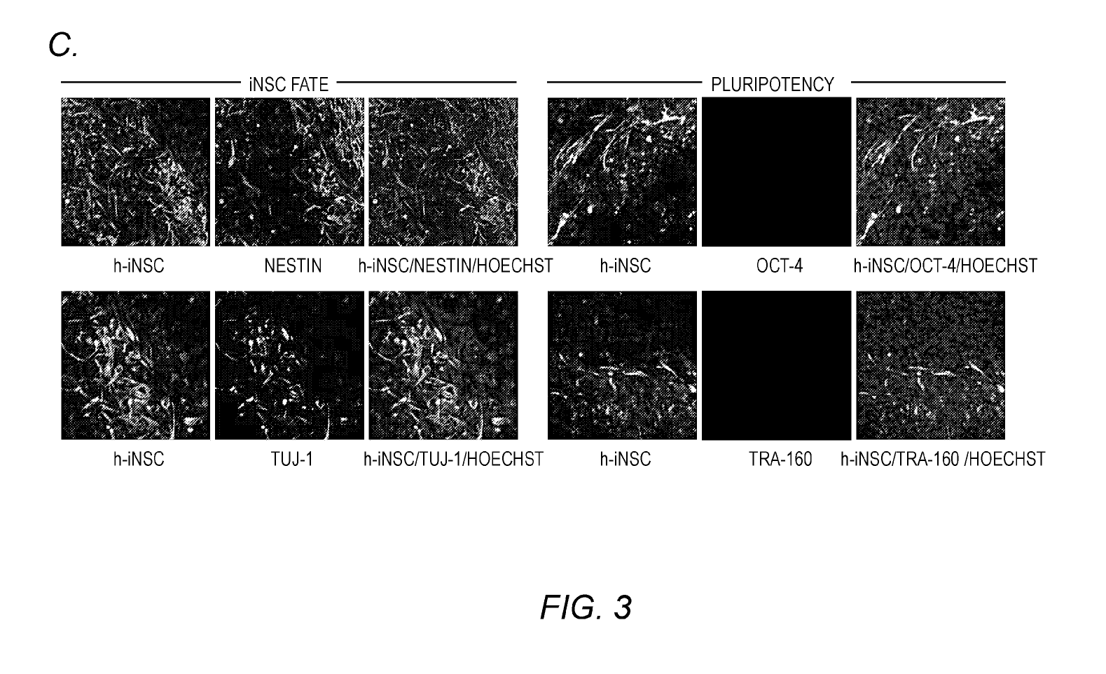

We next utilized the engineered h-iNSCs to investigate the survival and fate of these cells in vivo in the brain. A previous study of in vitro proliferation after engineering of h-iNSC with GFPFL and mCFL showed no significant differences with non-engineered h-iNSCs (FIG. 3A). For in vivo study, h-iNSCs engineered with mCFL was stereotactically implanted in the brain of mice and real-time non-invasive imaging was used to monitor cell survival over time. Capturing images periodically, we found that h-iNSCs survive more than 20 days post implantation (FIG. 3B). Post-mortem IHC revealed that approximately half of h-iNSC-mCFL expressed the NSC marker nestin (FIG. 3D) and the other half were positive for the neuronal marker Tuj-1 (FIG. 3D). No astrocyte marker GFAP was observed. Additional IHC verified the transplanted h-iNSCs did not express the pluripotency markers Oct-4 and TDR-160.

Efficacious Treatment of Malignant and Invasive GBM Using Tumoricidal iNSCs.

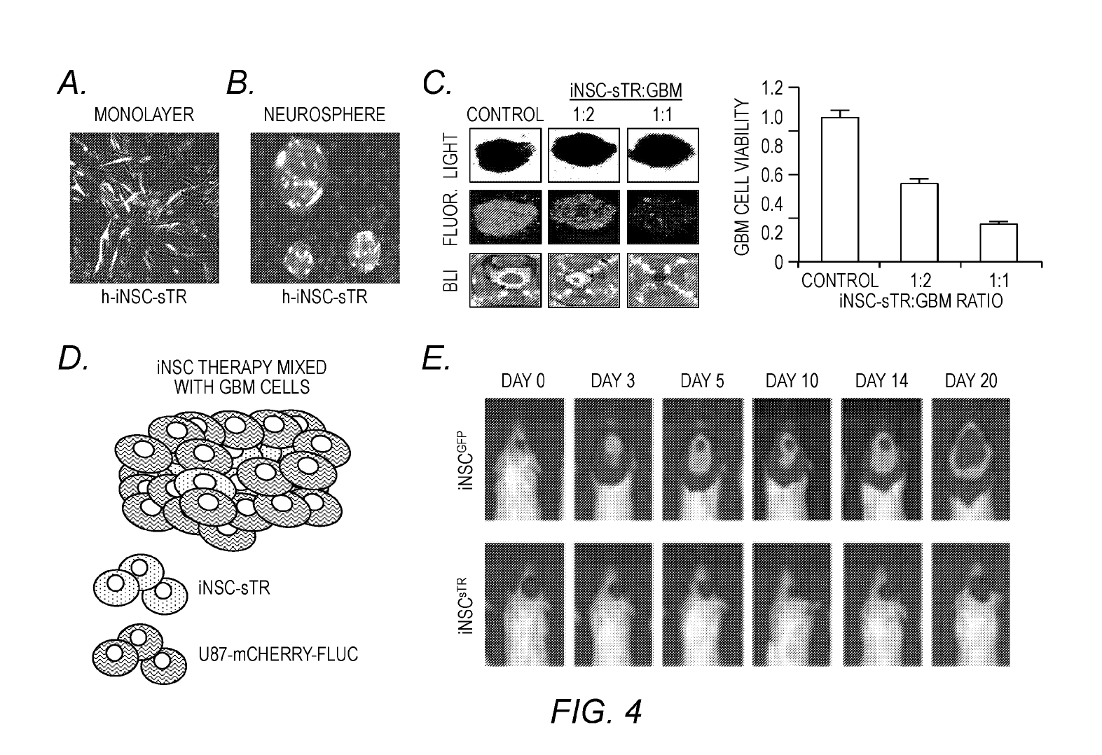

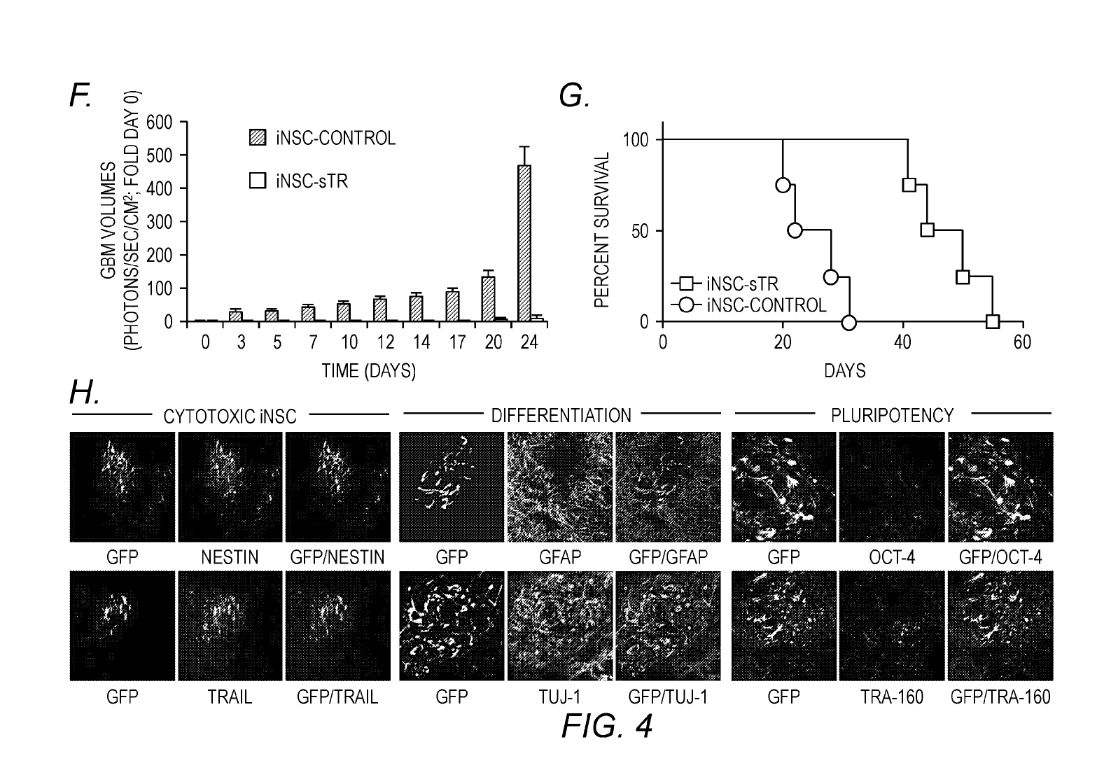

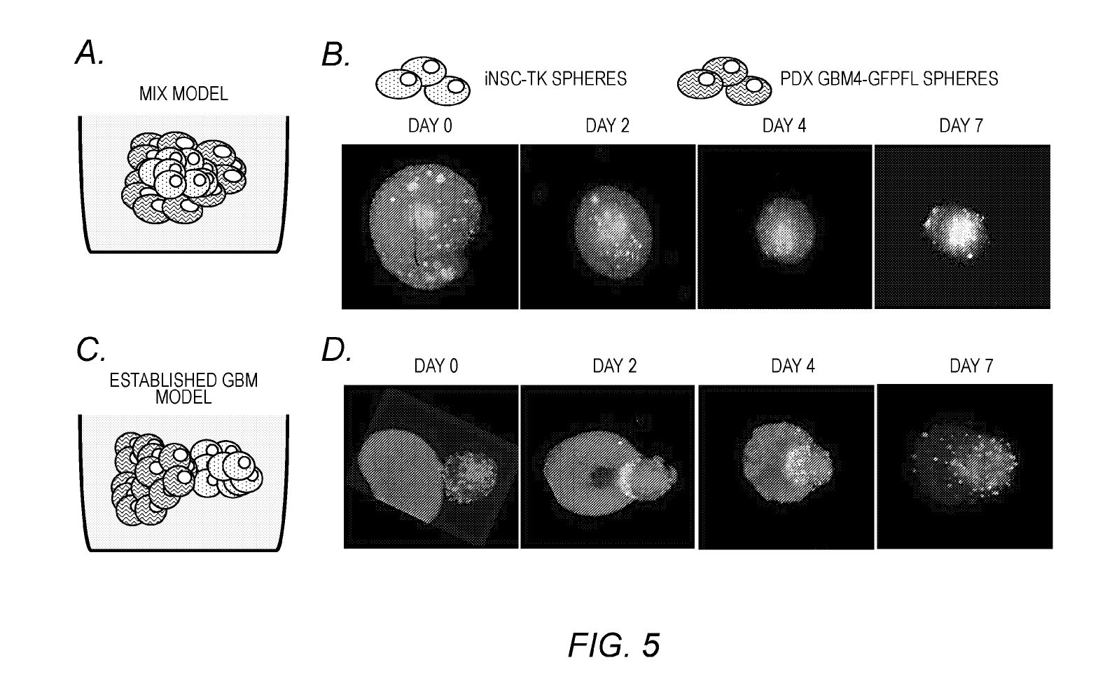

To investigate the therapeutic efficacy of h-iNSC-based GBM treatment, we first engineered h-iNSCs to express a secreted variant of the pro-apoptotic molecule TNFα-related apoptosis-inducing ligand (TRAIL; diTR) in frame with Gaussia luciferase and upstream of an IRES-GFP element (iNSC-diTR). Anti-cancer effects of TRAIL when delivered from engineered cell carriers were established previously; therefore it is the ideal tumoricidal molecule for characterizing new h-iNSC delivery vehicles. Robust expression of the GFP reporter was detected following transduction of the h-iNSCs (FIG. 4A). We observed that h-iNSC-diTR efficiently formed neurospheres when cultured in suspension (FIG. 4B), and displayed proliferative capacity and passage numbers equivalent to unmodified cells (data not shown). Nestin expression and differentiation capacity were the same as observed in previous engineered and not engineered h-iNSC, suggesting that modification of h-iNSCs with TRAIL does not interfere with their properties as stem cells.

To evaluate the anti-GBM efficacy of engineered h-iNSCs, h-iNSC-diTR or control iNSC-GFPRL were co-cultured at different ratios with human GBM cells expressing mCherry and firefly luciferase (mC-FL). In order to mimic the in vivo characteristics, GBM and h-iNSC were mixed and cultured in three-dimensional levitation system for 48 hours. Fluorescence and BLI revealed a significant reduction in the viability of GBMs co-cultured with h-iNSC-sTR. This reduction was significantly greater if a higher h-iNSC:GBM ratio was used (FIG. 4C).

h-iNSC Secretion of a Pro-Apoptotic Agent Reduces Solid GBM.

To test the in vivo efficacy of h-iNSC-sTR based therapy, we determined the effects of h-iNSC-sTR treatment on solitary human GBMs. Human U87 GBM cell expressing mC-FL were implanted intracranially with iNSC-sTR or control iNSC-GFP (Fig. D) and tumor volumes were followed using serial bioluminescence imaging. We found that h-iNSC-sTR treatment induced a statistically significant reduction in tumor growth by day 3 and decreased GBM volumes 50-fold by day 24 (FIG. 4F). In addition, h-iNSC-sTR-treated animals survived more than 51 days, while control animal succumbed to GBM growth in only 25 days (FIG. 4G). IHC examination of mouse brains showed a robust expression of TRAIL by the h-iNSC-sTR after two weeks. The h-iNSC-sTR in the GBM were positive for the expression of the Nestin and Tuj-1, and negative for GFAP and pluripotency markers Oct-4 and TRD-160 (FIG. 4H).

Efficacious Treatment of Malignant and Invasive GBM CD133+ Using Tumoricidal iNSCs.

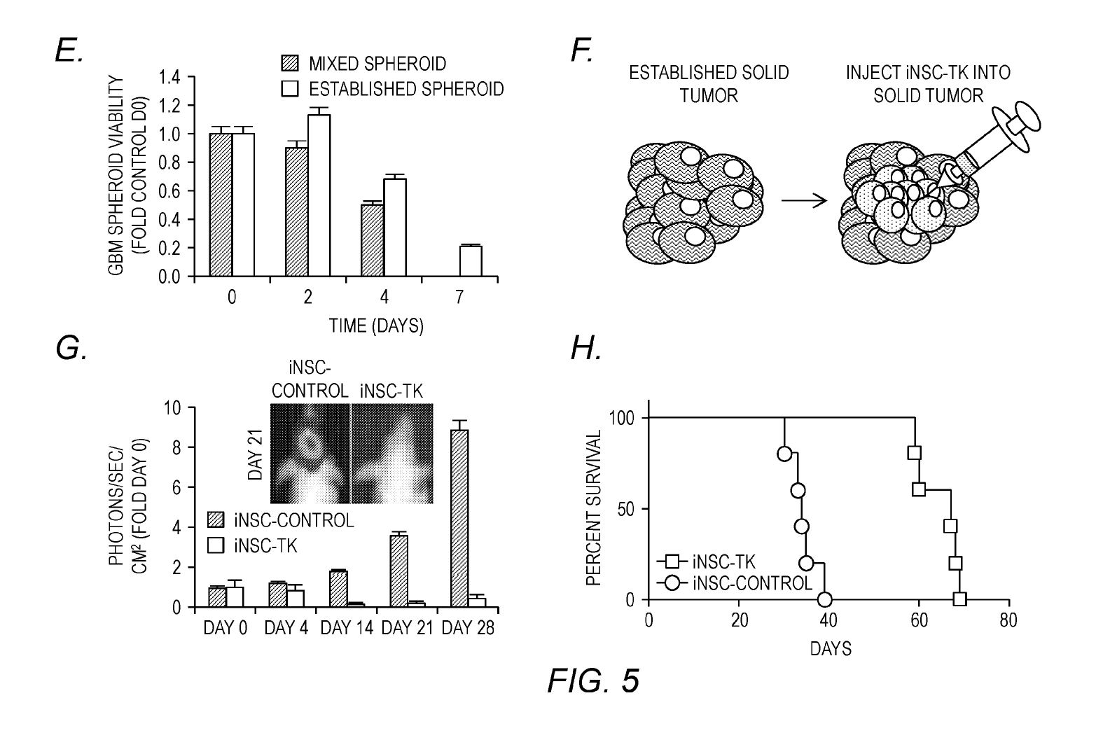

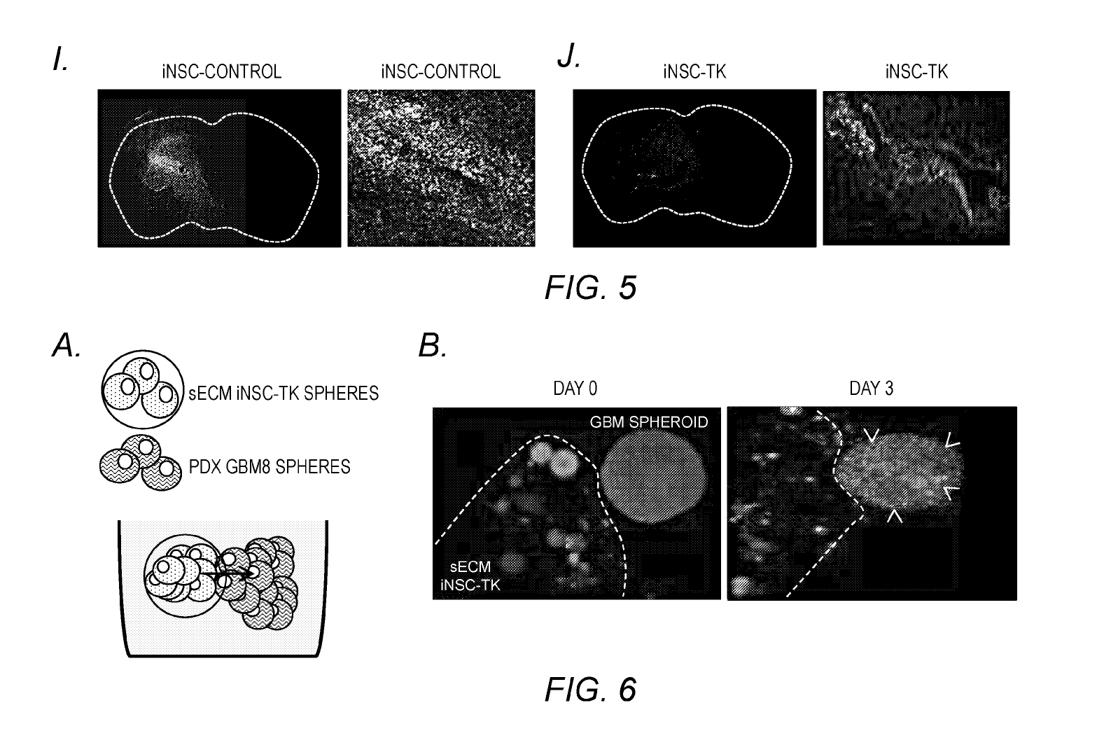

To determine the efficacy of h-iNSC prodrug/enzyme therapy for patient-derived CD133+ human GBM-initiating cells, we co-cultured GBM4 cells expressing GFP and firefly luciferase (GBM4-GFPFL) with h-iNSC expressing a trifunctional chimeric reporter including Rluc, RFP and thymidine kinase (TK) activities, to generate h-iNSC-TK. The thymidine kinase encoded by herpes simplex virus (HSV-TK) was used in the first cell suicide gene therapy proof of principle and still is one of the most widely used systems in clinical and experimental applications. GBM4-GFPFL and h-iNSC-TK were co-cultured in three-dimensional levitation system in two different models. The first model (FIG. 5A) the two cell types were mixed and cell survival monitored over time by fluorescence (FIG. 5B). The second model, the two cell types were cultured side by side to mimic the treatment of an established GBM (FIG. 5C). Cell survival was monitored over time by fluorescence (FIG. 5D). In both cases, a significant reduction of the GBM survival was observed over time, being more significant in the mixed model (FIG. 5E).

We next determined the efficacy of h-iNSC-TK therapy in vivo on established GBM4 by implanting GBM4-GFPFL cancer cells into the parenchyma of mice. Three days later, h-iNSC-TK or control cells were administered directly into the established tumors (FIG. 5F). Serial bioluminescence imaging showed that h-iNSC-TK treatment attenuated the progression of GBM4 tumors, reducing tumor burden by 9-fold compared to control 28 days after injection (FIG. 5G). h-iNSC-TK therapy also led to a significant extension in survival as h-iNSC-TK treated animals survived an average of 67 days compared to only 37 days in control-treated mice (FIG. 5H). Post-mortem IHC verified the significant reduction in tumor volumes by h-iNSC-TK injection (FIG. 5I-5J). Together, these results show that h-iNSC-TK therapy has significant therapeutic effects against malignant and invasive GBM and markedly prolongs the survival of tumor-bearing mice.

Intracavity h-iNSC-TK Therapy Inhibits Surgically Resected GBM Recurrence.

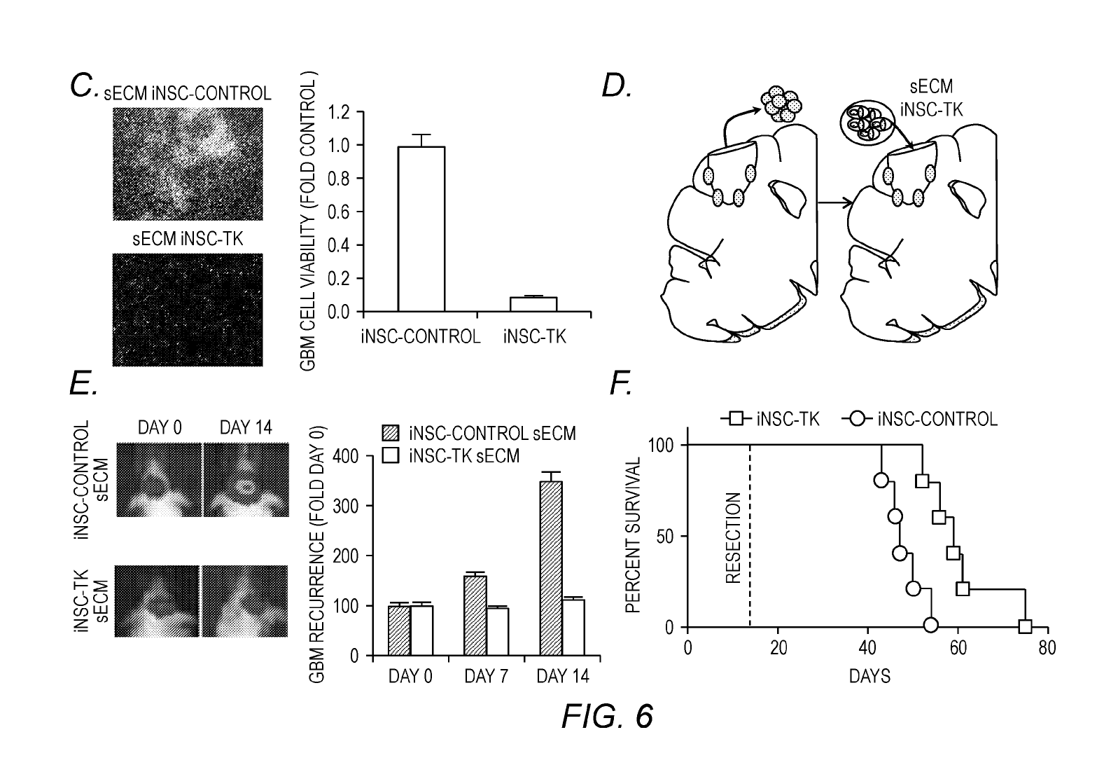

Surgical resection is part of the clinical standard of care for GBM patients. We previously discovered that encapsulation of stem cells is required for intracavity therapy to effectively suppress GBM recurrence. To determine the efficacy of h-iNSC therapy encapsulated in synthetic extracellular matrices (sECM), we co-cultured GBM-8 GFPFL (patient-derived CD133+ human GBM-initiating cell) with h-iNSC-TK embedded in HLA hydrogels (FIG. 6A). We found that mCherry+h-iNSCs migrated from the sECM matrix and populated GFP+ GBM8 spheroids within 3 days. Additionally, sECM/h-iNSC-TK therapy dramatically reduced the viability of GBM8 spheroids in 3 days.

To mimic h-iNSC therapy for surgically resected human GBM patients, we tested h-iNSC-TK therapy against highly diffuse patient-derived GBM8 cells in a mouse model of GBM resection (FIG. 6D). h-iNSC-TK embedded in HLA were transplanted into the surgical resection cavity following GBM debulking Serial bioluminescence imaging showed that h-iNSC-TK therapy attenuated the recurrence of GBM8 tumors, reducing tumor burden by 350% compared to control 14 days after implantation (FIG. 6E). h-iNSC-TK therapy also led to a significant extension in survival as h-iNSC-TK treated animals survived an average of 59 days compared to 46 days in control-treated mice (FIG. 6E).

### Example 2: Alternative Media for Rapid Transdifferentiation of Human Skin Cells

Transdifferentiation of human skin cells was performed as above in Example 1, but in place of the STEMdiff™ Neural Progenitor Basal Medium was a 1:1 mixture of N-2 medium and B-27 medium as follows. Chemicals were purchased from Gibco® (Invitrogen Corporation, Carlsbad, Calif.), Sigma (Sigma-Aldrich, St. Louis, Mo.) or Selleck Chemicals (Houston, Tex.) as indicated.

### N-2 Medium:

### DMEM/F12 (Gibco®)

1×N2 supplement (Gibco®)
5 μg/ml insulin (Sigma)

### 1 mM L-glutamine (Gibco®)

### 1 mM Glutamax (Gibco®)

100 μM MEM non-essential amino acids (NEAA) (Gibco®)
100M beta-mercaptoethanol (bME)

### B-27 Medium:

Neurobasal medium (Gibco®)
1×B-27 supplement (Gibco®)

### 200 mM L-glutamine (Gibco®)

To the 1:1 mix was added bovine serum albumin (BSA, Sigma) to a final concentration of 5 μg/ml.

This medium was supplemented with the following: SB431542 (Selleck Chemicals) to a final concentration of 10 μM; LDN193189 (Selleck Chemicals) to a final concentration of 100 nM; all trans retinoic acid to a final concentration of 10 μM (Sigma); and CHIR99021 (Selleck Chemicals) to a final concentration of 3 μM.

Using this media, nestin+ iNSCs were generated when used with the Sox2 transduction.

The medium may also be made to include Insulin (25 μg/ml), Transferrin (100 μg/ml), Sodium selenite (30 nM), and/or cAMP (100 ng/ml).

### Example 3: Use of iNSCs in Treatment for Brain Cancer

A patient is diagnosed with brain cancer (e.g., glioblastoma), and surgery is scheduled for removing the tumor soon thereafter (e.g., within one, two or three weeks). A skin punch is taken from the patient to obtain skin fibroblast cells. The cells are transdifferentiated as taught herein into induced neural stem cells and also loaded with a therapeutic agent and/or a reporting molecule. During surgery and after removal of the tumor, the loaded iNSCs are administered into the resulting cavity where the tumor had been removed. The iNSCs migrate toward residual cancer cells and deliver their therapeutic agent/reporting molecule payload.

The foregoing is illustrative of the present invention, and is not to be construed as limiting thereof. The invention is defined by the following claims, with equivalents of the claims to be included therein.

1. A method for making an induced neural stem cell, comprising:
providing a somatic cell;
transfecting or transducing said somatic cell with a nucleic acid encoding Sox2, whereby said somatic cell expresses Sox2; and then
transdifferentiating said somatic cell, wherein said transdifferentiating is carried out by growing said somatic cell in a neural progenitor medium,
to thereby make said induced neural stem cell.

2. The method of claim 1, wherein said transdifferentiating is carried out for a time of from 1 to 10 days.

3. The method of claim 1, wherein said transdifferentiating is carried out for a time of from 1 to 5 days.

4. The method of claim 1, wherein said transdifferentiating is carried out for a time of from 1 to 3 days.

5. The method of claim 1, wherein said somatic cell is a fibroblast cell.

6. The method of claim 1, wherein said somatic cell is a skin fibroblast cell.

7. The method of claim 1, wherein said induced neural stem cell expresses nestin and/or does not express Nanog or OCT3/4.

8. The method of claim 1, wherein said induced neural stem cell is capable of differentiating into a neural/astrocyte cell.

9. The method of claim 1, wherein said method comprises transducing said somatic cell with a lentiviral vector comprising said nucleic acid encoding Sox2.

10. The method of claim 1, wherein said transdifferentiating is carried out without feeder cells.

11. The method of claim 1, wherein said neural progenitor medium comprises one or more ingredients selected from the group consisting of: DMEM/F12; N2 supplement; insulin; neurobasal medium; B27 supplement; non-essential amino acids; bME; L-glutamine; Glutamax; SB431542; LDN193189; retinoic acid; BSA; CHIR99021; transferrin; sodium selenite; and cAMP.

12. The method of claim 1, wherein said method further comprises transfecting or transducing said somatic cell with a nucleic acid encoding TERT (e.g., hTERT).

13. The method of claim 1, wherein said method further comprises loading said induced neural stem cell with a therapeutic agent and/or a reporter molecule.

14. The method of claim 13, wherein said loading comprises transfecting or transducing said induced neural stem cell with a nucleic acid capable of producing a therapeutic agent, and wherein said therapeutic agent is a protein toxin, an oncolytic virus, a pro-apoptotic agent, or an enzyme useful for enzyme/prodrug therapy.

15. The method of claim 14, wherein said nucleic acid capable of producing a therapeutic agent and said nucleic acid encoding Sox2 are provided on the same vector.

16. The method of claim 1, wherein said method further comprises expanding said induced neural stem cell to form a population of induced neural stem cells.

17. The method of claim 1, wherein said method further comprises encapsulating said induced neural stem cell in a hydrogel or seeding said induced neural stem cell onto a scaffold.

18. The method of claim 1, wherein said method further comprises administering said induced neural stem cell to a subject in need thereof.

19. The method of claim 18, wherein said induced neural stem cell is allogeneic, syngeneic or autologous with respect to said subject.

20. The method of claim 18, wherein said subject is a human subject.

21. The method of claim 18, wherein said subject is in need of treatment for a brain cancer.

22. The method of claim 21, wherein said brain cancer is glioblastoma.

23. The method of claim 21, wherein said administering is carried out by intracavity administration after surgical removal of at least a part of a brain tumor.

24. The method of claim 18, wherein said induced neural stem cell is autologous with respect to said subject and wherein said administering is carried out 1 to 14 or 21 days after said providing the somatic cell.

25. An induced neural stem cell produced by a process comprising:
providing a somatic cell;
transfecting or transducing said somatic cell with a nucleic acid encoding Sox2, whereby said somatic cell expresses Sox2; and then
transdifferentiating said somatic cell, wherein said transdifferentiating is carried out by growing said somatic cell in a neural progenitor medium,
to thereby make said induced neural stem cell.

26. The induced neural stem cell of claim 25, wherein said transdifferentiating is carried out for a time of from 1 to 10 days.

27. The induced neural stem cell of claim 25, wherein said induced neural stem cell expresses nestin and does not express Nanog or OCT3/4.

28. The induced neural stem cell of claim 25, wherein said somatic cell is from a patient in need of treatment for a brain cancer.

29. The induced neural stem cell of claim 25, wherein said method further comprises loading said induced neural stem cell with a therapeutic agent and/or a reporter molecule.
